(19) United States
(12) Patent Application Publication
(10) Pub. No.: US 2006/0004313 A1
(43) Pub. Date: Jan. 5, 2006

(54) Cured body shell and method of making the same

(76) Inventors: Thomas J. Heinz, La Canada, CA (US); Royce Rumsey, Laguna Beach, CA (US); Eddie C. Bannister, Victorville, CA (US); William L. King, Tehachapi, CA (US)

Correspondence Address: ARENT FOX PLLC, 1050 CONNECTICUT AVENUE, N.W., SUITE 400, WASHINGTON, DC, 20036, US

(21) Appl. No.: 11/157,160

(22) Filed: Jun. 21, 2005

Related U.S. Application Data
(60) Provisional application No. 60/581,364, filed on Jun. 22, 2004.

(51) Int. Cl. A61F 5/00

(52) U.S. Cl. 602/7; 602/5; 602/6; 602/8

(57) ABSTRACT

A custom-fitted orthotic device including a rigidizable panel emplaceable on a body part. A padding member is mounted on an inner surface of the curable panel between the panel and the body part. A fastening system is attached to an outer surface of the curable panel to secure the orthotic device on the body part.

This application claims priority to applicants' co-pending U.S. Provisional Application Ser. No. 60/581,364 entitled “CURED BODY SHELL AND PROCESS” filed Jun. 22, 2004. The entirety of this patent application is incorporated herein by reference.

### BACKGROUND OF THE INVENTION

1. Field of the Invention

The present invention relates to an orthotic device, and more specifically, relates to a customized orthotic device formed on a body part for limiting movement, or providing support to a portion of the body.

2. Description of the Related Art

Orthotic devices are used to support, align, prevent, or correct deformities or to brace weak or ineffective joints or muscles. The problems with existing custom-fitted orthotic devices include the high cost and the length of time required to construct the product. In particular, there is generally a lengthy delay, typically days, between the time that the user is packed in the plaster so that a model can be made, the product is formed and the user is fitted. Refitting is often required. The present invention solves the problems of delay, re-fitting and the high cost of a custom-fitted orthotic by allowing an orthotist to construct the device in one fitting.

### SUMMARY OF THE INVENTION

The custom-fitted orthotic device of the present invention includes a curable panel emplaceable on a body part. The curable panel is a flexible panel that is impregnated with, for example, a curable resin that, upon curing, causes the flexible panel to become rigid. The rigid panel, when applied to a body part, supports, aligns, prevents, or corrects deformities or braces weak or ineffective joints or muscles. The device can include a padding member mounted on an inner surface of the rigid panel or shell, between the rigid panel and the body part to protect the skin from any roughness of the rigid panel. This orthotic device can also incorporate a fastening or closure system attached to an outer surface of the rigid panel to hold the orthotic device on the body or to supply the proper amount of support to the body part.

The present invention also includes a method of making a custom-fitted orthotic device for treating a body part. The method includes mixing or combining a fibrous material with a resin, such as a liquid resin, to form a flexible panel. The flexible panel can then be applied to the body part and molded to the shape of the body part. The flexible panel can then be cured in place on the body part, or removed from the body part and then cured.

The flexible-to-rigid panel of the present invention can be formed of an assembly of mechanical parts emplaceable on a body part. The mechanical parts include, for example, interlocking rings, interwoven coils, panel of interlocking sections, a series of overlapping plates, an assembly of snap-fitted molded parts. The flexible-to-rigid panel can also be formed from a corrugated panel having a honeycomb configuration. Each of the different panels include a curable resin or adhesive that allows the mechanical parts of the assembly to rigidly join with each other as a thermoset body.

Additional aspects, features, and advantages of the invention will be set forth in the description which follows, and in part will be apparent from the description, or may be learned by practice of the invention. The aspects, features, and advantages of the invention will be realized and attained by the structure and steps particularly pointed out in the written description, the claims, and the drawings.

### BRIEF DESCRIPTION OF THE DRAWINGS

The features of the invention will be more readily understood with reference to the following description and the attached drawings, wherein:

FIG. 1 illustrates a front view of the panel in accordance with the present invention;

FIG. 2 illustrates a cross-sectional view of the panel in accordance with the present invention;

FIGS. 3A and 3B illustrate one method of curing and securing the panel in accordance with the present invention;

FIGS. 4A and 4B illustrate a front view and cross-sectional view, respectively, of a portion of the panel in accordance with a second embodiment of the present invention;

FIG. 5 illustrates a perspective of a portion of the panel in accordance with a third embodiment of the present invention;

FIG. 6 illustrates a front perspective view of a portion of the panel in accordance with a fourth embodiment of the present invention;

FIGS. 7A and 7B illustrate a front view and an exploded partial view respectively, of a portion of the panel in accordance with a fifth embodiment of the present invention;

FIGS. 8A and 8B illustrate a perspective view and cross sectional view, respectively, of a portion of the panel in accordance with a sixth embodiment of the present invention;

FIG. 9 illustrates a perspective view of a portion of the panel in accordance with a seventh embodiment of the present invention;

FIG. 10 illustrates the curing of one embodiment of the flexible panel in accordance with the second embodiment of the present invention;

FIGS. 11A and 11B illustrate the flexible panel in accordance with the eighth embodiment of the present invention;

FIG. 12 illustrates a sectional view of FIG. 11A;

FIG. 13 illustrates a perspective view of the flexible panel and padding in accordance with a ninth embodiment of the present invention; and

FIG. 14 illustrates a sectional view of FIG. 13.

### DETAILED DESCRIPTION OF INVENTION

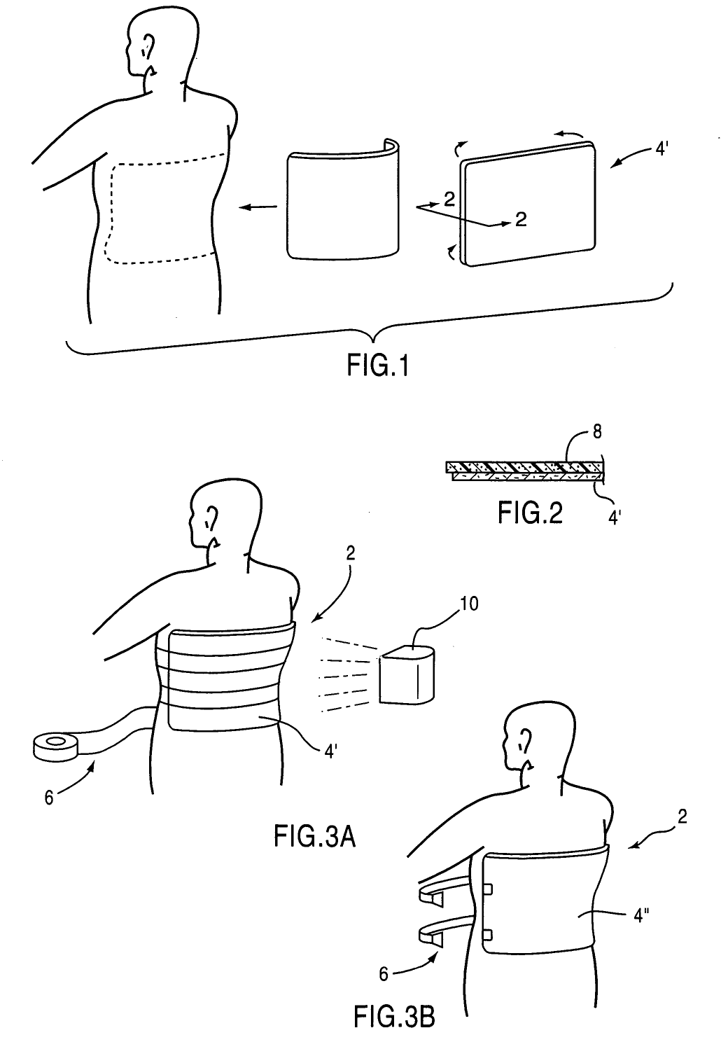

The present invention is directed to a custom-fitted orthotic device for applying pressure to hold a body part in a constrained position, thus limiting movement of the body part, and/or supporting the body part. For example, as shown in FIG. 1, the custom-fitted orthotic device 2 is in the form of a cured or rigid panel, designated by reference numeral 4″, for supporting the back of a user. The custom-fitted orthotic device 2 of the present invention also includes a padding member mounted on an inner surface of the shell, and a fastening system for holding the shell on the body part.

The present invention is also directed to a method of making the custom-fitted orthotic device 2. The rigid panel 4″ of the orthotic device 2 is preliminarily formed as an uncured or fibrous panel, designated by reference numeral 4′, that can be soft, flexible and conformable fibrous material impregnated with an uncured substance in the form of, for example, a liquid, a powder, or co-mingled fibers. In one embodiment of the present invention, the uncured substance is an uncured resin. See FIG. 2. The fibrous material can include any flexible or semi-flexible material, such as, for example, fiberglass, various metals, plastic, wood, paper, pulp, hemp or hemp-like material, and other natural fibers, synthetic fibers, or hybrids thereof. Such hybrids of materials include combinations of metal and fiberglass, or wood and paper. The fibrous material can be woven, knit, felted or stitched to form a substantially flat sheet. The fibers in the fibrous material could be randomly oriented and held in place by the viscosity of the uncured resin. The fibrous material of the flexible panel 4′ can be supplied to an orthotist as a pre-cut shape or as a bulk material, such as a roll of material that can be cut to a desired shape.

The resin dispersed throughout the fibrous material can be cured while the flexible panel 4′ is mounted onto the body part and shaped to conform to the body part. Then, the flexible panel 4′ can undergo curing to become a rigid panel 4″ or shell that is rigid to the contour of the body part. The resin can also be cured before the shell is mounted onto the body part. As such, the shell can be shaped to a desired contour before it is mounted onto the body part.

The resin can comprise any chemical composition that has the ability to change state from a liquid or a gel into a solid. The resin can be polyester, vinylester, epoxy, acrylic, urethane resins, a combination of resins, or a combination of thermoset resins combined with thermoformable resins.

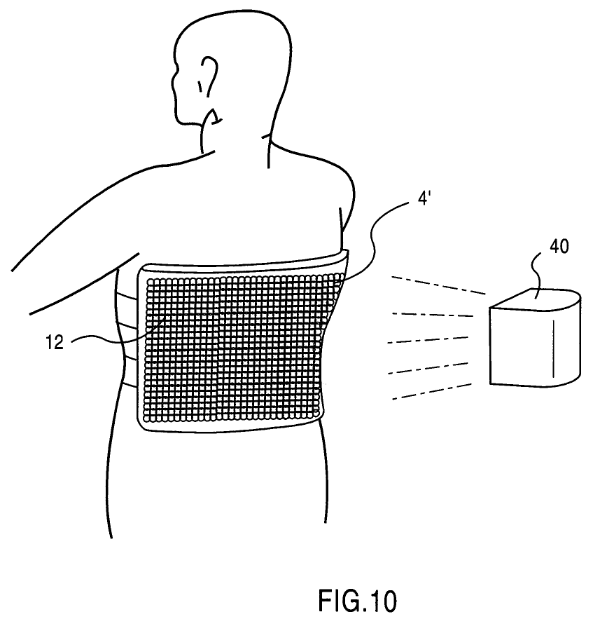

FIG. 1 illustrates an example of the shell 4. In this embodiment, the shell is a half-shell that is applied to and conformed to the back of the user. The shell can include a similar construct that is applied to and conformed to the front of the user, or the shell can include a single wraparound body having a variable opening at the front or back side. After the desired shape is created, the shell can be cured so that it becomes rigid. This flexible-to-rigid transformation can be effected in a number of ways including, but not limited to the application of heat, moisture, a solvent, mechanical means or photo-initiated means. For example, in the fibrous material that is cross-linked with the liquid resin, the resin can harden at room temperature or a higher temperature, such as in an oven. Depending on the chemical composition of the resin, the curing time can range from several minutes to several hours. In a preferred embodiment, a light-curable, UV-curable or acrylic resin can be used, as illustrated in FIG. 10.

At the start of the curing process, a resin, such as a liquid resin, dispersed within the fibrous material is made to harden to a gelatinous or semi-liquid state so that the resulting flexible panel 4′ becomes firm so that it can be easily and accurately cut to size. The resin can be heated in, for example, in an oven. The heated resin creates a warm shell that can be malleable for molding. The warm shell is applied to the body part so that the shell can conform to the body part as it cures. The shell can be heated to a temperature above room temperature, up to around 140 degrees Fahrenheit, which is the upper temperature limit of human comfort. At the end of the curing process, the resin reaches a cured state, by which it can be easily cut. The resulting shell is thermoset because, when cured, it is irreversibly rigid and impervious or relatively unaffected by heat.

As noted above, the fibrous material can be impregnated with a powder resin. The powder resin includes granules of plastic that adhere to one another in the presence of a solvent or heat and are held in place on the fibrous material by electrostatic cling, or mechanical or physical constraints such as, for example, a covering. The mixed fibrous material and powder resin forming a flexible panel 4′ can be cured to the rigid panel 4″ or a shell after first softening the powder therein with a solvent, such as water, or melting the powder by the addition of heat. Softening or melting the powder in the fibrous material causes the granules to adhere to one another, resulting in a plaster-like cast that can be applied to the body part whereby the powder resin hardens or becomes crystalline.

The fibrous material can also be impregnated with a low-temperature resin that cures at room temperature. Such a flexible panel 4′ can be shipped and stored at a low temperature, such as 40 degrees Fahrenheit or below. As shown in FIG. 3A, the fibrous material of the flexible panel 4′ can also be impregnated with a compound having a photo-initiated curing chemistry that reacts upon exposure to light energy 10 of a selected wavelength. The fibrous material impregnated with a photo-initiated curing chemistry can be transported in light-impervious containers and removed from the containers when the desired shape is achieved. Exposure of the resin to light energy 10 in a predetermined spectrum, for example, 360-400 nm can cause cross-linking of the resin and the fibrous material resulting in the rigid panel 4″.

The resins in the flexible panel 4′ also be fabricated from materials such as co-mingled fibers, which include, for example, glass and other fibers, such as that found in polyester fiberglass. Threads of extruded fiberglass are woven into a construct formed from being woven together with plastics such as, but not limited to, polyester and nylon. The cloth fibers can be heated to a melting point, or softened with a solvent to a liquid state, allowing the cloth fibers to adhere to the fiberglass. The cloth fibers then re-solidify at room temperature or higher to give the flexible panel 4′ the rigid construct of a shell.

The flexible panel 4′ can be configured any number of ways. For example, the flexible panel 4′ can be an assembly of smaller linked mechanical parts that are bonded together to become rigid.

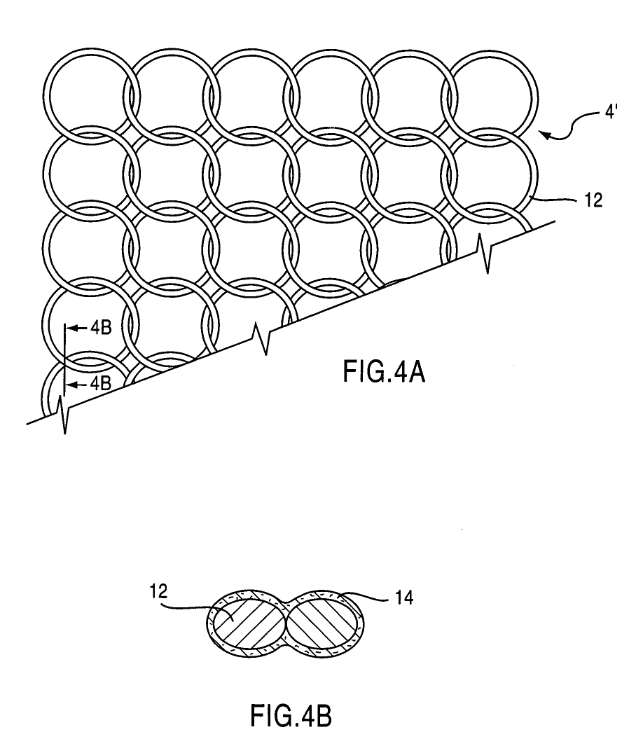

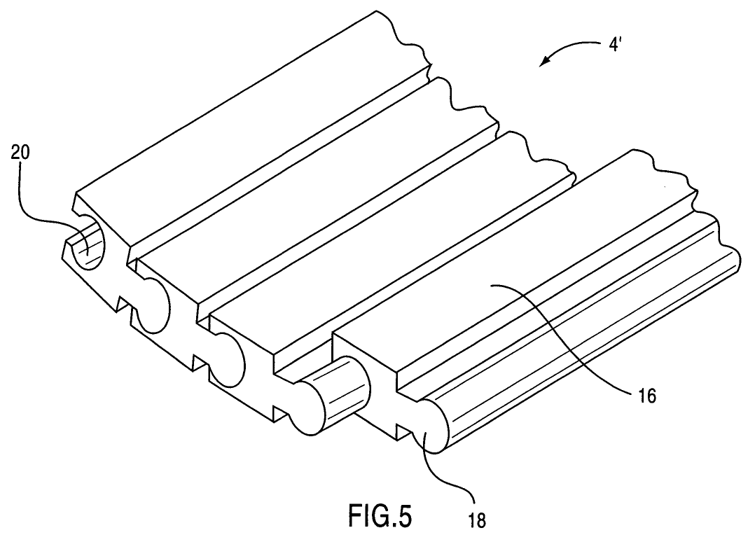

The present invention also includes a custom-fitted orthotic device 2 including a flexible-to-rigid or curable assembly of mechanical parts emplaceable on a body part. The assembly of mechanical parts is flexible until they adhere to each other. For example, as shown in FIG. 4A, the flexible panel 4′ can be formed from an assembly of interlocking rings 12, similar to chain mail, can be used. These rings can be plastic or an extruded fibrous material coated with adhesive 14. When exposed to solvent or vapors or heat, the adhesive coating 14 softens, releasing adhesive properties and the adhesive 14 in the coatings on adjacent rings flow together, as shown in FIG. 4B. As a result, after cooling, the assembly forms a rigid construct. As such, the step of curing can also include applying a reactant 40 to the rings to activate the adhesive. The reactant 40 can be introduced by means of a vapor, or sprayer, or brushing with or without the addition of heat.

In another embodiment of the present invention, the flexible panel 4′ can be formed as a panel of interlocking sections 16. The interlocking sections 16, as shown in FIG. 5 can be plastic extruded sections having a tongue 18 on one side and a channel 20 on an opposite side. Adjacent sections are slidingly connected in a lateral direction by their respective tongue and channel. The interlocking sections 16 can slide along the channels of adjacent sections and pivot relative to each other. When the flexible panel 4′ is cured, the tongues and adjacent channels adhere to each other so that the panel becomes rigid.

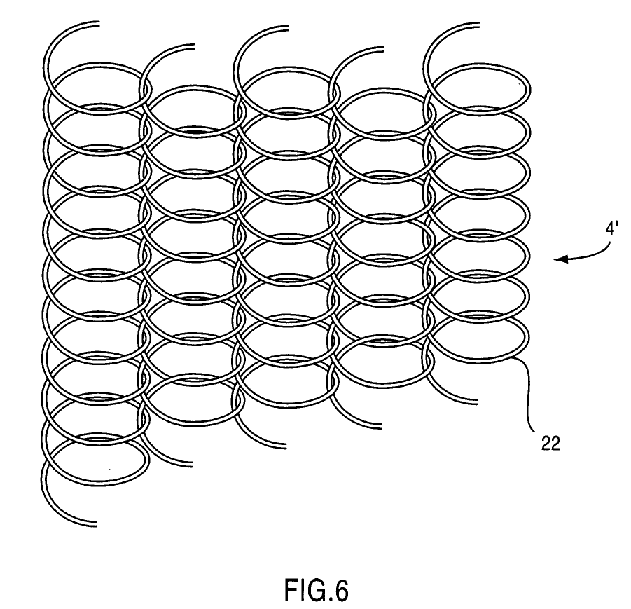

In another embodiment of the present invention, the flexible panel 4′ can also be formed as an assembly of interlocking coils 22, similar to a chain link fence as shown in FIG. 6. The plastic or extruded fibrous material that forms the interlocking coils can be cured in the same manner as the interlocking rings 12 discussed above.

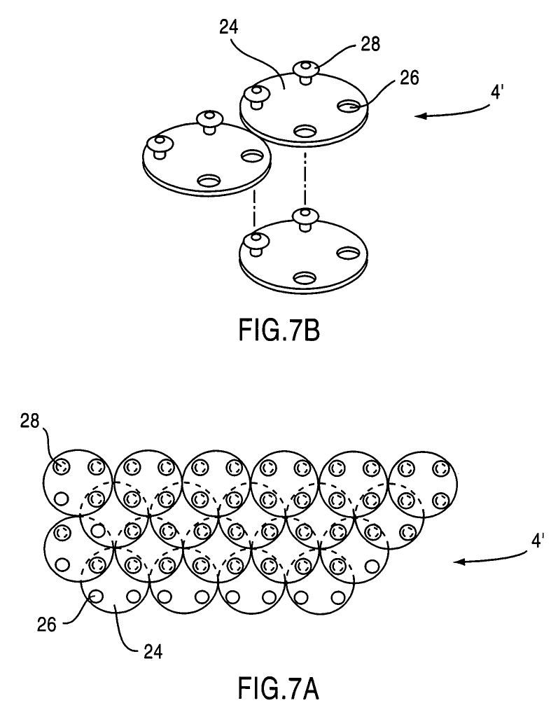

In another embodiment of the present invention, the flexible panel 4′ can be formed as a series of overlapping plates 24 adhering to the padding member or loosely sewn together, similar to fish scales, as shown in FIG. 7A. The plates 24 can be formed from plastic or any material that softens in the presence of heat or a solvent and thereby be cured to a rigid fit. As shown in the exploded view of FIG. 7B, each plate can include a plurality of holes 26. When the plates are overlapped and one or two of the holes 26 of a first plate are aligned with holes of a second plate, a post 28 can be inserted into the aligned holes. The posts 28 can be formed of the same material as the plates 24, for example, plastic. The assembled plates can form a flexible sheet because of the loose fit between the posts 28 and holes 26. See FIGS. 7A and 7B. The sheet can be cured to a rigid construct by applying a reactant such as a solvent or heat to the plastic sheet causing the plates and posts adhere to each other.

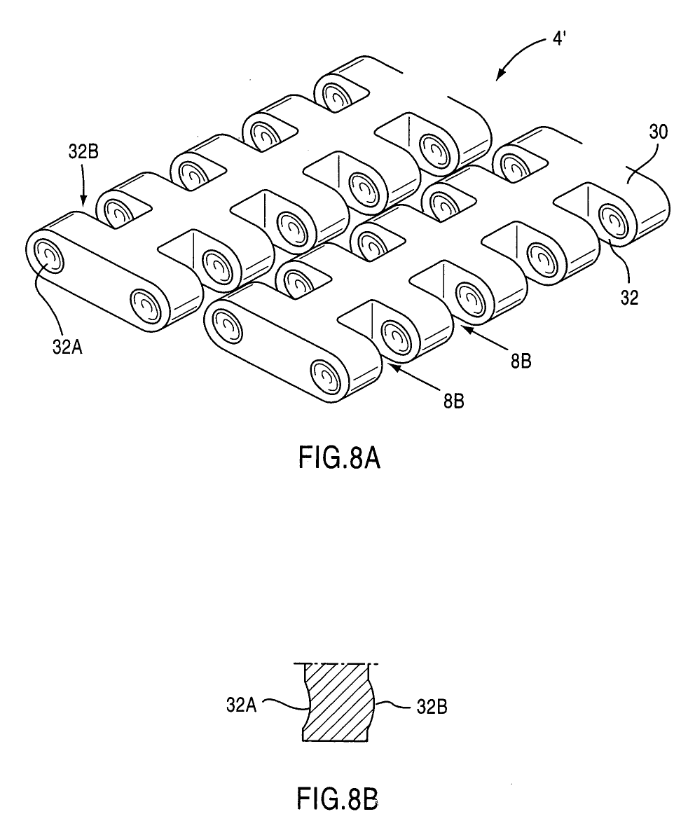

In another embodiment of the present invention, shown in FIGS. 8A and 8B, the flexible panel 4′ can be formed from an assembly of molded components 30 having snap fitting sections 32 that join the components 30. The snap fitting sections are molded on stalks so that each stalk can snap into an adjacent stalk. A plurality of stalks are snapped together form the an assembly that constitutes the flexible panel 4′. Upon curing, the adhesive coating on the plurality of stalks softens allowing adjacent stalks adhere to each other. When the assembly cools, the flexible panel 4′ becomes the a rigid panel 4″.

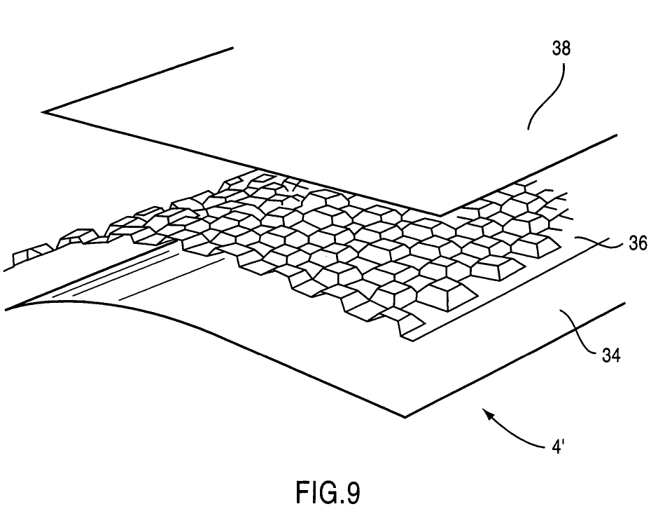

The flexible-to-rigid panel can also be formed by joining layers of materials similar to corrugated cardboard or a honeycomb as shown in FIG. 9. A flexible inner sheet 34 of material is placed against and conformed to the body part. The material can include any cloth, plastic sheet of any size suitable for the particular user. A “core” of material 36 is then adhered to this inner sheet 34. This “core” 36 can be paper or plastic material has a plurality of protrusions resulting in a thickness greater than a of the material. The protrusions can resemble corrugations or a honeycomb type of core, or corrugations running at right angles to each other. An outer sheet of material or cover 38 can be adhered to the core to protect and seal the core. In addition, the cover 38 protects the skin of user, and keeps the rigid panel from scratching or sticking to surfaces external of the user and provides an aesthetic look to the orthotic device 2. The flexible panel 4′ can be made in different sizes tailored to different users and their respective body parts so that only minor shape adjustments are necessary in order to obtain the desired form. For example, flexible panel 4′ can have a semi-circular shape that approximates the shape and size of a portion of the body part. The flexible panel 4′ can be manipulated to an appropriate fit and then cured.

As shown in FIG. 10, the resin in the flexible panel 4′ can be cured by spraying a curing agent 46 or cross-linking material from a sprayer 40 against the flexible panel, or exposing the flexible panel to vapors, for example water and alcohol.

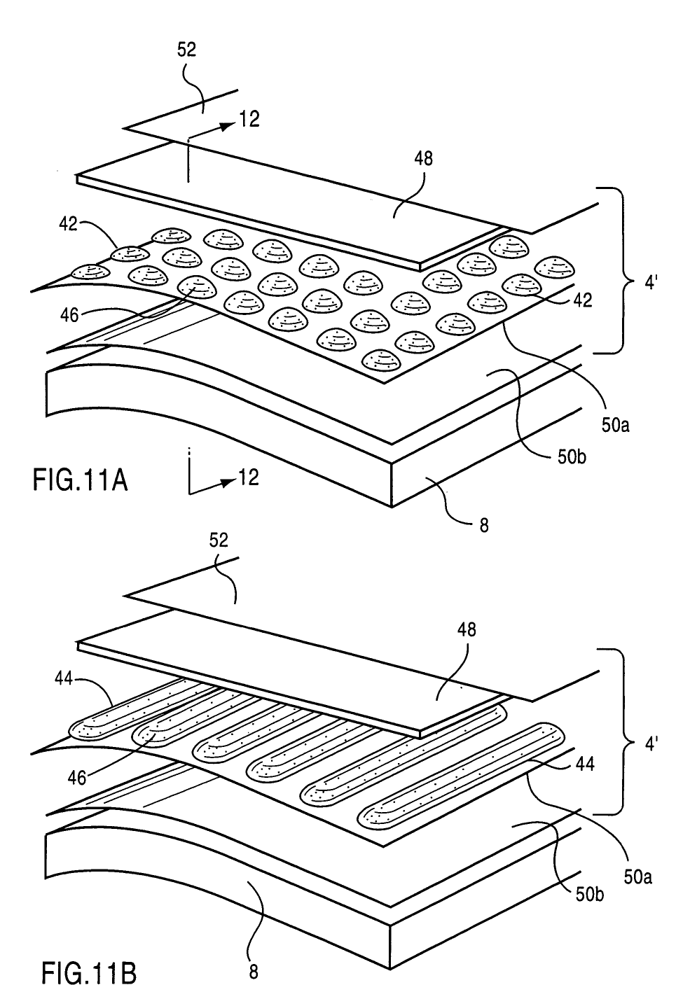

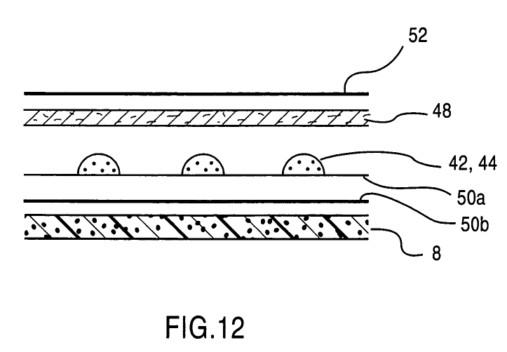

As shown in FIGS. 11A and 11B, the present invention includes a pre-form for use in making the custom-fitted orthotic device 2. The pre-form includes a fibrous material uncured resin member 48, a frangible material 42, and the padding 8.

A curing agent or cross-linking material can be incorporated into the frangible material, such as, for example, a plurality of frangible bladders 42 fixed in a member having an upper portion 50a and a lower portion 50b. The lower portions 50b helps retain the curing agent 46 in the upper portion. The member can be formed of, for example, a polymeric material, or any suitable material for retaining the curing agent, and can have a shape similar to that of bubble wrap. The frangible bladders 42 can be fractured by locally-applied pressure. The bladders 42 can have any size, such as, for example a ¼″ diameter and higher, or long bubble tubes 44 that extend the length of the flexible panel 4′. An outer sheet of material or cover 52 can be placed adjacent to a substantially flat fibrous material and uncured resin member 48 to protect and seal the uncured contents of the panel 4′. The cover can be formed of any material including, for example, a polyvinyl alcohol (PVA) film. The cover 52 allows the orthotist to move the fibrous material uncured resin mixture within the flexible panel, without the mixture being in direct contact with the orthotist's hands. In the fibrous material uncured resin member 48, the fibers in the fibrous material can be held in place by the viscosity of the uncured resin. When the frangible bladders 42, 44 burst, the curing agent 46 is released to react with the resin in the member 48, thereby forming the rigid panel 4″. The cover 52 also protects the skin of user, and keeps the rigid panel from scratching or sticking to surfaces external of the user and provides an aesthetic look to the orthotic device 2. The bladders 42, 44 may release their curing or cross-linking material 46 by the application of energy, such as radio frequency waves, light energy (in the visible spectrum or invisible spectrum, such as ultraviolet light), or by heat energy, such as, for example, about 160 degrees Fahrenheit for up to approximately ten minutes. Heat energy can be supplied by, for example, hot air, a conductive blanket, radiant heat, or by body heat. The frangible bladders 42, 44 can also release their curing or cross-linking material 46 through the application of pressure. The release of the cross-linking material 46 may be performed immediately before the flexible panel 4′ is placed on the body part or after the flexible panel 4′ is on the body part.

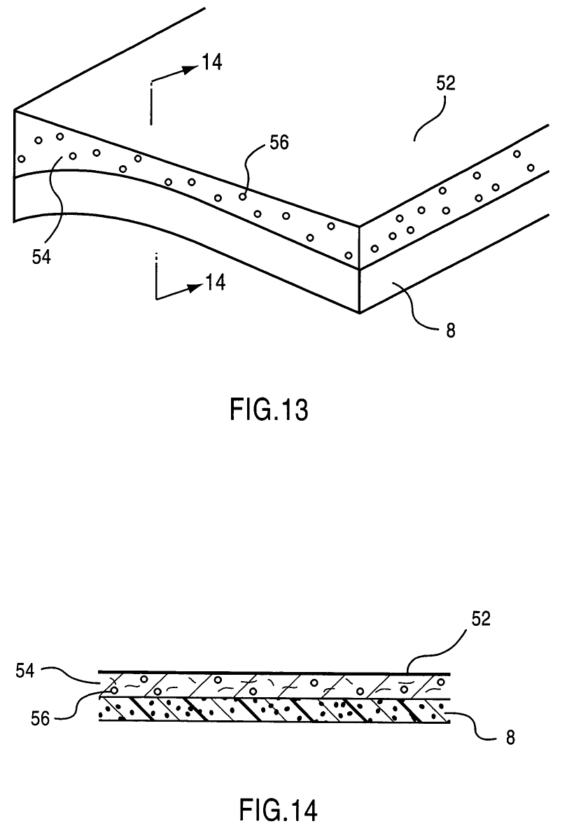

In another embodiment of the present invention, a pre-form for use in making a custom-fitted orthotic device includes a cover 52, a fibrous material and resin mixture 54, a frangible material 56, and a padding 8 as shown in FIGS. 13 and 14. As discussed above, the flexible panel 4′ can be formed by a loose mixture of the fibrous material and uncured resin 54 into which is dispersed frangible bubbles 56 freely movable within the resin and fibrous mixture. The resin in the fibrous material is cured by releasing a curing agent 46 encapsulated in frangible bubbles 56. The fibrous material of the flexible panel 4′, is impregnated with an uncured resin, forming a mixture that can adhere to the padding 8 by the viscosity of the uncured resin. See FIG. 14. The encapsulated curing agent 46 can be released into the fibrous material uncured resin mixture 54 by methods including, but not limited to breaking the bubbles 56 containing the curing agent 46 by manipulation of the flexible panel 4′. For example, a small diameter, handheld roller can be rolled over the flexible panel 4′ surface, causing local deformation sufficient to release the curing or cross-linking agent. In addition, The bubbles 56 may release their curing or cross-linking material 46 by the application of energy, such as radio frequency waves, light energy (in the visible spectrum or invisible spectrum, such as ultraviolet light), or by heat energy for example, of about 160 degrees Fahrenheit. Heat energy can be supplied by, for example, hot air, a conductive blanket, radiant heat, or by body heat.

The present invention also includes a method of making a custom-fitted orthotic device 2 for treating a body part. The method includes mixing or combining a fibrous material with a resin to form a flexible panel 4′. The flexible panel 4′ is then applied to the body part where it is molded to the shape of the body part. The flexible panel 4′ is then cured by any one or more of a number of means including the application of heat, ultraviolet light or other light energy 10, a conductive blanket or the application of an external reactant 40 such as by spraying the flexible panel 4′, or releasing or activating a curing agent or reactant from within the flexible panel. The activation of an internal reactant can include applying pressure, heat energy, or light energy to the flexible panel to release an encapsulated curing agent or cross-linking material from frangible bubbles or bladders located in the resin.

The application of heat to the flexible panel 4′ makes the flexible panel malleable for molding. The flexible panel 4′ can be heated and molded either before or after it is placed on the body part. Heating includes heating the flexible panel 4′ to a to a gelatinous state by a temperature of up to 140 degrees Fahrenheit, which is the upper limit of human comfort. The flexible panel 4′ heated prior to placement on the body part can be heated in an oven.

As shown in FIG. 2, the padding member 8 is mounted on an inner surface of the rigid panel between the rigid panel and the body part. The padding member 8 can be joined with the rigid panel 4″ at the time of curing or separate from the rigid panel and added to the completed custom-fitted orthotic device 2 after the rigid panel 4″ is formed.

The padding member 8 can be formed from an impermeable material, or it can be formed from a breathable or vapor permeable material. The material can be a foam or combination of materials. This combination may include a cloth or fibrous cover (such as felt) over foam padding. The cloth cover can be made removable for washing.

The foam padding can be natural or synthetic rubber, or a plastic material such as polyester, polyethylene, polyurethane, silicone or neoprene. Many of these foam padding materials can be reticulated to make a breathable material.

Semi-rigid foam padding can be used such as a sheet of foam beads that are adhered together such that the spaces between the beads allow the passage of air or vapor. This foam is rigid enough to be thermoformed to the desired shape. With the panel attached to the body shape as a flexible member, the assembly can be fitted and then the panel hardened.

The fastening system 6 of the present invention includes, but is not limited to a wrapping member wound around the rigid panel 4″ as shown in FIG. 3A, or a belt and a pulley system. The fastening system 6 is attached to an outer surface of the rigid panel 4″ to secure the orthotic device 2 on the body part.

An adjustable belt can be physically or mechanically fixed to the rigid panel 4″. The pulley system includes at least one or more closure mechanisms that are located on the sides and front of the rigid panel 4″; the pulley system being centrally attached to the rigid panel 4″. During the curing process a receptacle can be molded into the rigid panel 4″ to hold the pulley system in place, or a hook and loop arrangement, such as, for example, Velcro®, rivets or screws. Any of the assemblies shown in the FIGS. 4-9, can have protuberances for the attachment of the pulley-tightening system can be attached.

An alternative fastening system includes an elastic member laterally extendable across the body as shown in FIG. 3B. The elastic member has clips on at least one end, which fasten onto the rigid panel 4″, similar to suspenders.

In a preferred embodiment of the present invention, a rigid panel 4″ of a fiberglass material is impregnated with a light energy curable resin and adheres to a sheet of padding by the viscosity of the uncured resin. The padding member 8 is held in place with a light energy transparent film or wrap. The resin is cured with light-emitting lamp. It is sometimes possible to control the cure of the resin to allow trimming to shape with scissors before final cure. As discussed above, the process of curing the resin can include the application of heat, moisture, a solvent, mechanical means and photo-initiated means. Light energy also can cause the resin to undergo a cationic curing process in which curing continues to occur even in the resin shaded from light energy.

Example embodiments of the present invention have now been described in accordance with the above advantages. It will be appreciated that these examples are merely illustrative of the invention. Many variations and modifications will be apparent to those skilled in the art.

We claim:

1. A custom-fitted orthotic device comprising:
a curable panel emplaceable on a body part;
a padding member mounted on an inner surface of the curable panel; and
a fastening system attached to an outer surface of the curable panel to secure the orthotic device on the body part.

2. The custom-fitted orthotic device according to claim 1, wherein the curable panel comprises a thermoset shell.

3. The custom-fitted orthotic device according to claim 1, wherein the padding member is vapor permeable.

4. The custom-fitted orthotic device according to claim 1, wherein the fastening system comprises a belt and pulley system.

5. The custom-fitted orthotic device according to claim 1, wherein the curable panel is formed from a fibrous material impregnated with a resin.

6. The custom-fitted orthotic device according to claim 5, wherein the curable panel is formed from a material selected from the group consisting of fiberglass, resin, metal, plastic, wood, paper, pulp, and hemp.

7. The custom-fitted orthotic device according to claim 2, wherein the curable panel comprises a plurality of interlocking rings coated with an adhesive.

8. The custom-fitted orthotic device according to claim 2, wherein the curable panel comprises a plurality of interlocking sections.

9. The custom-fitted orthotic device according to claim 2, wherein the curable panel comprises a plurality of linked coils.

10. The custom-fitted orthotic device according to claim 2, wherein the curable panel comprises a plurality of overlapping plates.

11. The custom-fitted orthotic device according to claim 2, wherein the curable panel comprises a plurality of snap-fitted molded parts.

12. The custom-fitted orthotic device according to claim 2, wherein the curable panel comprises a corrugated panel.

13. The custom-fitted orthotic device according to claim 5, wherein the resin is selected from the group consisting of polyester, vinylester, epoxy, acrylic, urethane resin and combinations thereof.

14. The custom-fitted orthotic device according to claim 5, wherein the resin comprises a powder resin having granules of plastic that adhere to each other in the presence of a solvent or heat.

15. The custom-fitted orthotic device according to claim 5, wherein the fibrous material impregnated with a resin comprises a curing agent encapsulated in bubbles within a mixture of the fibrous material and resin.

16. A method of making a custom-fitted orthotic device for treating a body part, the method comprising:
mixing a fibrous material with a resin to form a flexible panel;
applying the flexible panel to the body part;
molding the flexible panel to the shape of the body part; and
curing the flexible panel to form a rigid panel.

17. The method according to claim 16, wherein the step of applying comprises the step of heating the flexible panel to make the flexible panel malleable for molding.

18. The method according to claim 17, wherein the step of heating comprises heating the flexible panel to a temperature of up to 140 degrees Fahrenheit.

19. The method according to claim 17, wherein the step of further comprises heating the flexible panel in an oven.

20. The method according to claim 16, wherein the step of curing comprises hardening the resin to a gelatinous state.

21. The method according to claim 16, wherein the step of curing comprises activating a reactant in the flexible panel.

22. The method according to claim 16, wherein the step of curing comprises applying light energy to the flexible panel.

23. The method according to claim 16, wherein the step of curing comprises spraying a curing agent onto the flexible panel.

24. The method according to claim 16, wherein the step of mixing comprises incorporating a curing agent encapsulated within frangible bubbles into the fibrous material and resin mixture to form the flexible panel.

25. The method according to claim 24, wherein the step of curing comprises applying pressure to the flexible panel to release a curing agent.

26. The method according to claim 24, wherein the step of curing comprises applying heat energy to the flexible panel to release a curing agent encapsulated within the frangible bubbles.

27. A custom-fitted orthotic comprising:
a curable assembly of mechanical parts at least a portion of which are coated with an adhesive, emplaceable on a body part;
a padding member mounted on an inner surface of the curable assembly; and
a fastening system attached to an outer surface of the curable assembly to secure the orthotic on the body part.

28. The custom-fitted orthotic according to claim 27, wherein the curable assembly comprises an assembly of interlocking rings.

29. The custom-fitted orthotic according to claim 27, wherein the curable assembly comprises an assembly of interwoven coils.

30. The custom-fitted orthotic according to claim 27, wherein the curable assembly comprises a panel of interlocking sections.

31. The custom-fitted orthotic according to claim 27, wherein the curable assembly comprises a series of overlapping plates.

32. The custom-fitted orthotic according to claim 27, wherein the curable assembly comprises an assembly of snap-fitted molded parts.

33. The custom-fitted orthotic according to claim 27, wherein the curable assembly comprises a corrugated formed material and formable sheet material.

34. The custom-fitted orthotic according to claim 28, further comprising applying a reactant to the curable assembly to activate the adhesive.

35. The custom-fitted orthotic according to claim 34, wherein the reactant is selected from the group consisting of a solvent, a vapor, and heat.

36. A custom-fitted orthotic device for treating a body part, comprising:
supporting means for supporting a body part;
padding means for cushioning the body part supported by the supporting means, the cushioning means begin mounted on an inner surface of the supporting means; and
fastening means for fastening the supporting means to the body part.

37. The custom-fitted orthotic device according to claim 36, wherein the supporting means comprises a thermoset shell.

38. The custom-fitted orthotic device according to claim 36, wherein the padding means comprises vapor permeable foam.

39. The custom-fitted orthotic device according to claim 36, wherein the fastening means comprises a belt and pulley system.

40. A pre-form for use in making a custom-fitted orthotic device comprising:
a cover;
a fibrous material and resin mixture adjacent to the cover;
a padding; and
a frangible material between the padding and the mixture.

41. A pre-form for use in making a custom-fitted orthotic device comprising:
a fibrous material and resin member;
a padding; and
a frangible material between the fibrous material and the resin member.
